(19) United States
(12) Patent Application Publication
(10) Pub. No.: US 2023/0402193 A1
(43) Pub. Date: Dec. 14, 2023

(54) SYSTEMS AND METHODS FOR SPATIAL ENTROPYOMICS FOR THE TREATMENT OF CANCER

(71) Applicant: Kambiz Afrasiabi, Anaheim, CA (US)

(72) Inventor: Kambiz Afrasiabi, Anaheim, CA (US)

(21) Appl. No.: 18/148,354

(22) Filed: Dec. 29, 2022

Related U.S. Application Data
(60) Provisional application No. 63/366,373, filed on Jun. 14, 2022.
(60) Provisional application No. 63/374,304, filed on Sep. 1, 2022.
(60) Provisional application No. 63/377,313, filed on Sep. 27, 2022.

(51) Int. Cl. G16H 70/60; C12Q 1/6886

(52) U.S. Cl. CPC G16H 70/60; C12Q 1/6886; C12Q 2600/106

(57) ABSTRACT

Systems and methods for developing a spatial entropyomic profile of a neoplasm are contemplated, and further include the administration of therapies based upon the developed spatial entropyomic profile in order to treat neoplasms by targeting the region of the neoplastic mass identified as the driver zone of tumor growth. Through the novel concepts of spatial entropyomics, a present-state driver zone of neoplastic growth within a tumor may be identified, and from such identification, targeted therapies may be developed and administered. Further, from evolutionary projections, candidate future-state driver zones of the neoplastic mass which may potentially develop into active future-state driver zones may also be identified, which enables the creation and administration of therapies to inhibit the potential of candidate future-state driver zones to develop into active future-state driver zones, or to prophylactically treat projected active future-state driver zones prior to any detection thereof.

### CROSS-REFERENCE TO RELATED APPLICATIONS

This patent application claims the benefit and priority of U.S. Provisional Patent Application No. 63/366,373, entitled NANOMACHINES FOR CANCER TREATMENT, filed on Jun. 14, 2022, and claims the benefit and priority of U.S. Provisional Patent Application No. 63/374,304, entitled PROGRAMMABLE NANOMACHINES FOR TREATMENT OF CANCER, filed on Sep. 1, 2022, and claims the benefit and priority of U.S. Provisional Patent Application No. 63/377,313, entitled SPATIAL ENTROPYOMICS, filed on Sep. 27, 2022, the disclosure of each of which are incorporated by reference herein in their entirety as part of the present application.

### STATEMENT RE: FEDERALLY SPONSORED RESEARCH/DEVELOPMENT

Not Applicable

### BACKGROUND

### 1. Technical Field

The present disclosure relates generally to the field of cancer therapies. More specifically, the present disclosure relates to systems and methods for treating cancer based upon principles of cellular network entropy and nanomachinery.

The evolution of thought and design of cancer therapeutics has gone through a tortuous path in the last eighty or so years, ever since the first patient was treated with a chemical agent. Introduction of nitrogen mustard in the treatment of cancer in 1940's was based on a serendipitous finding and observation during the Second World War. Design of multiagent chemotherapy protocols which was the next step in the evolution of this path, was based on the understanding that killing cancer cells through interference with their life cycle in multiple directions and through multiple paths, would lead to a better outcome. Through time, dissection of different intracellular pathways related to cell survival and proliferation has guided the design of newer agents interfering with and antagonizing those pathways.

The driving force of conventional thinking has thus essentially been development of more sophisticated means to kill cancer cells. Recurrence of cancer following complete response, as well as upfront resistance to chemo and targeted therapy has led to significant frustration and roadblock in clinical arena. This has led to consideration of other kinds of approach to neoplastic disorders.

The second law of thermodynamics has rightfully been recognized as the most fundamental law that prevails the known universe. Interplay of living cell with this law is perhaps the most fascinating of all. The most elegant part of this interplay is to maintain the network entropy of numerous subcompartments of living cell at the lowest possible level, dictated by the limits of the second law. This inversely correlates with the maximum amount of free energy in different subcompartments of the living cell. During the lifetime of living organisms, there is a constant tug of war, which at one end drags the cell towards higher level of entropy through the spontaneous increase in entropy of the surrounding universe, and a pullback by the constituents of living cell to lowest possible level, at the other end.

While cancer has conventionally been conceived of as a disease to be eradicated, it is also important to recognize that it in many ways represents a thermodynamic process of evolution on the microscopic and micro-environmental level, whereby the cells most able to survive and proliferate thrive, even if the unchecked replication of such cells is ultimately dysfunctional in view of the organism in which the cells reside.

Therefore, it is important to develop new approaches for understanding cancer and in developing therapeutics for preventing and treating cancer.

### BRIEF SUMMARY

To solve these and other problems, systems and methods for developing a spatial entropyomic profile of a neoplasm are contemplated, and thereafter administering therapies based upon the developed spatial entropyomic profile in order to treat the neoplasm by targeting the region of the neoplastic mass identified as the driver zone of tumor growth. In particular, it may be seen that through the novel concepts of spatial entropyomics as described herein, methods for determining a present-state driver zone of neoplastic growth within a tumor are enabled. In addition, methods are also disclosed for identifying, from evolutionary projections, candidate future-state driver zones of the neoplastic mass which may potentially develop into active future-state driver zones. Thus, it may be seen that the concepts of spatial entropyomics as described herein may also enable the creation and administration of therapies to inhibit or potentially interdict the potential of candidate future-state driver zones to develop into active future-state driver zones, or to prophylactically treat projected active future-state driver zones prior to any detection thereof.

According to an exemplary embodiment of the methods described herein, a method for developing a spatial entropyomic profile of a neoplasm is contemplated, the method comprising the steps of: obtaining at least one biopsy from a neoplasmic mass; analyzing at least one biopsy to derive a present state neoplasmic profile of the neoplasmic mass comprising at least one of: a genomic profile, an epigenetic profile, a micro-RNA network profile, a proteomic profile; identifying, from the present-state neoplasmic profile, a present-state driver zone of the neoplasmic mass, the present-state driver zone being the region of the neoplasmic mass displaying the highest cellular master regulator complex network entropy; and identifying, from an evolutionary projection of the present-state neoplasmic profile, one or more candidate future-state driver zones of the neoplasmic mass, a candidate future-state driver zone comprising a region of the neoplasmic mass having an elevated propensity to develop into a projected active future-state driver zone comprising a region of the neoplasmic mass displaying the highest cellular master regulator complex network entropy; wherein the height of the cellular master regulator complex network entropy of a given particular region of a neoplasmic mass is determined according to the magnitude of and confluence, relative to other regions of the neoplasmic mass, of a plurality of factors in that region including: elevated cellular entropy, elevated chromosomal instability, elevated intratumor heterogeneity, depressed transmembrane potential, elevated resistance to microenvironmental noxions to growth, elevating resistance to available pharmaceutical modalities, elevated resistance to survival inhibitory factors.

It is also contemplated that the above-described method may further comprise the step of administering a first therapy operative to reduce the master regulator network entropy of the present-state driver zone. The first therapy may comprise, or may be administered by, a programmable nano-machine.

The first therapy may comprise a broad range of potential therapies effective to reduce the master regulator network entropy of the present-state driver zone. For example, but without limitation, the first therapy may comprise the delivery of a micro-RNA to the present-state driver zone via a non-pathogenic virus. The first therapy may also comprise modification of the transmembrane potential of the cells within the present-state driver zone. The first therapy may also comprise physical disruption of the present-state driver zone. The first therapy may additionally comprise a non-pathogenic virus containing crispr-CAS9 gene editing machinery. The first therapy may further comprise the delivery of a gene of interest loaded into a liposome-like particle having an avidity for one or more surface receptors of one or more cells within the present-state driving zone.

It is additionally contemplated that the above-described methods may further comprise the step of administering a second therapy operative to inhibit the potential of the candidate future-state driver zone to develop into an active future-state driver zone. Similarly, the second therapy may comprise, or may be administered by, a programmable nano-machine.

The second therapy may comprise a broad range of potential therapies effective to inhibit the potential of the candidate future-state driver zone to develop into an active future-state driver zone. For example, but without limitation, the second therapy may comprise the delivery of a micro-RNA to the candidate future-state driver zone via a non-pathogenic virus. The second therapy may also comprise modification of the transmembrane potential of the cells within the candidate future-state driver zone. The second therapy may also comprise physical disruption of the candidate future-state driver zone. The second therapy may additionally comprise a non-pathogenic virus containing crispr-CAS9 gene editing machinery. The second therapy may further comprise the delivery of a gene of interest loaded into a liposome-like particle having an avidity for one or more surface receptors of one or more cells within the candidate future-state driver zone.

It is further contemplated that the above-described methods may further comprise the step of prophylactically administering a third therapy operative to reduce the master regulator network entropy of the projected active future-state driver zone. Similarly, the third therapy may comprise, or may be administered by, a programmable nano-machine.

The third therapy may comprise a broad range of potential therapies effectively reducing the master regulator network entropy of the projected active future-state driver zone. For example, but without limitation, the third therapy may comprise the delivery of a micro-RNA to the projected active future-state driver zone via a non-pathogenic virus. The third therapy may also comprise modification of the transmembrane potential of the cells within the projected active future-state driver zone. The third therapy may also comprise physical disruption of the projected active future-state driver zone. The third therapy may additionally comprise a non-pathogenic virus containing crispr-CAS9 gene editing machinery. The third therapy may further comprise the delivery of a gene of interest loaded into a liposome-like particle having an avidity for one or more surface receptors of one or more cells within the projected active future-state driver zone.

Furthermore, systems for accomplishing the above-described methods or aspects thereof are also contemplated, including systems tangibly embodied by one or more programs of instructions executable by a data-processing apparatus to partially or wholly accomplish the various of the herein-discussed steps involved with developing a spatial entropyomic profile of a neoplasm, as well as therapeutics systems designed to accomplish the steps of administering therapies thereafter to the neoplasm based upon the developed spatial entropyomic profile.

### BRIEF DESCRIPTION OF THE DRAWINGS

These and other features and advantages of the various embodiments disclosed herein are better understood with respect to the following descriptions and drawings, in which:

FIG. 1A is an illustration of a tumor mass cell zone of a neoplasmic mass;

FIG. 1B is an illustration of a tumor mass cell zone of a neoplasmic mass showing distorted signatures;

FIG. 1C is a schematic diagram of various programmable nano-machines according to embodiments of the present disclosure;

FIG. 2 is a chart illustrating the tendency towards increased diversity of driver zones within a neoplasm over time;

FIG. 3 is a chart showing the evolutionary procession of successive driver zones within a neoplasm as new driver zones with heightened cellular network entropy overtake old driver zones;

FIG. 4 is a chart illustrating the tendency of cellular network entropy to increase over time as reflected by various factors;

FIG. 5 is a chart illustrating the various projected evolutionary paths which may be taken by a candidate future-state driver zone within a neoplasm and the placticity of interchangeability between STIC and TMC driver zones; and

FIG. 6 is a chart illustrating the general pattern of the increase of genetic diversity within a neoplasm over time, in the presence and absence of the treatment methods described herein.

### DETAILED DESCRIPTION

As is contemplated by the disclosure herein, systems and methods for developing a spatial entropyomic profile of a neoplasm are contemplated, and thereafter administering therapies based upon the developed spatial entropyomic profile in order to treat the neoplasm by targeting the region of the neoplastic mass identified as the driver zone of tumor growth. In particular, it may be seen that through the novel concepts of spatial entropyomics as described herein, methods for determining a present-state driver zone of neoplastic growth within a tumor are enabled. In addition, methods are also disclosed for identifying, from evolutionary projections, candidate future-state driver zones of the neoplastic mass which may potentially develop into active future-state driver zones. Thus, it may be seen that the concepts of spatial entropyomics as described herein may also enable the creation and administration of therapies to inhibit or potentially interdict the potential of candidate future-state driver zones to develop into active future-state driver zones, or to prophylactically treat projected active future-state driver zones prior to any detection thereof.

The second law of thermodynamics is sitting at the center of evolution of life on earth. By the virtue of this law, the index of instability or disorder of the known universe is incessantly on the rise. This correlates inversely with free energy of a closed thermodynamic system. It has long been recognized that the living cell is the most efficient machinery as far as capability to minimize the speed of rise in entropy is concerned. All the subcompartments of living cell have evolved and have been selected toward achievement of this goal. This ranges from quaternary structure of cellular proteins, to elasticity of cell membrane, regulatory gene mechanisms, RNA spliceosomes, micro-RNA network, and epigenome.

Cellular networks, ranging from G-protein coupled receptors which act as the radar of cellular energetics, modulating fair and balanced distribution of cellular energy, to proteasomes and ubiquitination machinery which eliminate old proteins characterized by their distorted quaternary structures which portend significant decrease in their plasticity or free energy, follow the same law. This further extends to fine harmony and alignment of constituents of Krebs cycle and ATP generating machinery of mitochondrion. Mitotic spindles, microtubules, kinetochores, centromere geometry and centrosomes, as well as telomeres are not exceptions to this rule.

The two major components of this harmony are sensors, which exist at virtually any subcompartment of the cell and executors, which have evolved to adopt the task of keeping cellular networks entropy at the lowest level, as per the limits of the second law of thermodynamics. Perhaps the most important of these sensors are the ones that sense thermodynamics arrow of time, which directly lead to aging and increase in cellular network entropy. Telomeres could be considered the masterpiece built into the genome in this regard. Their shortening following consecutive rounds of mitosis reflects the passage of time, which at a critical threshold activates apoptotic machinery to prevent catastrophic cellular events.

As perfect a machinery as living cell is, clearly it cannot escape minimal increase in network entropy, the accrual of which through time, leads to aging, disease, and demise. In this regard, neoplastic transformation could be considered a process in which increase in cellular networks entropy happens at a massively increased pace, such that the built-in cellular machineries cannot catch up with its repair, even if they have remained functional. Clearly sensors, executors, or a combination of both could have become dysfunctional in different scenarios and to different degrees.

In general, a neoplasm can be conceived of as having one or more Tumor Mass Cell (TMC) zones which function to generates tumor bulk, by replication and generation of pre-existing and new gene signatures. Stem-like tumor initiating cell (STIC) zones on the periphery of the TMC zones may also house stem-like initiating cancer cells. STIC cells in these zones may potentially reprogram themselves into new tumor mass cells following surgical resection, by a multitude of mechanisms, which may eventually lead to the formation of new tumor mass zone cells.

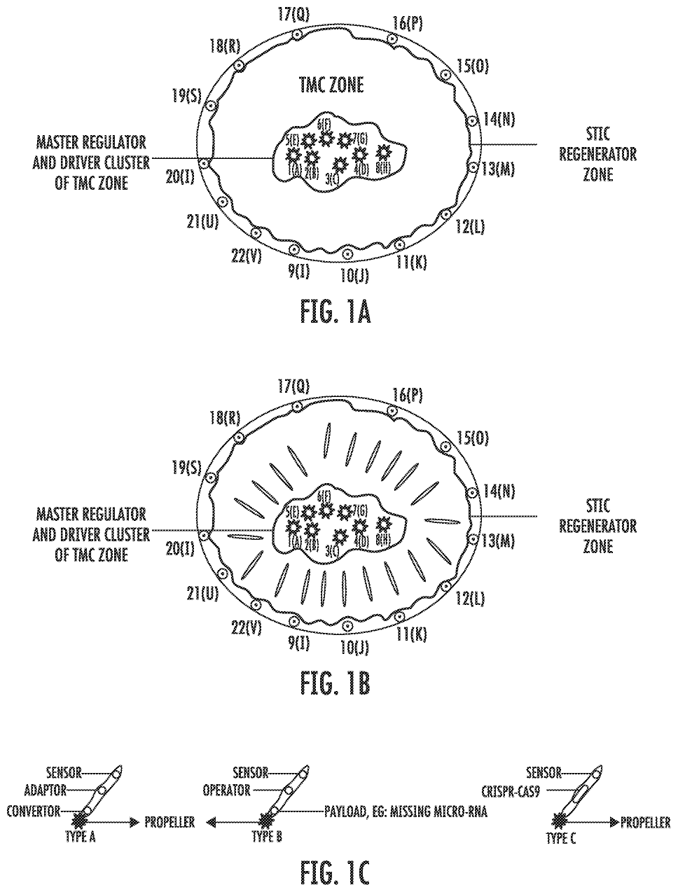

Turning now to FIG. 1, an illustration of a present-state TMC zone of a neoplasmic mass is shown. Clusters of master regulator and driver cells reside in TMC zones, and regenerator cells exist in STIC zones. Master regulator and driver cluster of cells in TMC zones may act as a driving forces for repopulating the tumor mass. Regenerator cells may also replenish the whole tumor mass with their diverse group of DNA, RNA, protein, and epigenome signatures following surgical resection. Elements 1(A) through 8(H) may be seen to correspond to different regions of driver cells (numerals 1-8) within the cluster and the corresponding master regulator complex network entropy values (letters A-H) they hold. Likewise, within the STIC regenerator zone, it may be seen that the clusters of stem-like initiating cells (numerals 9-22) may also hold their own corresponding master regulator complex network entropy values (letters I-V).

According to the presently disclosed methods, it is contemplated that a biopsy derived from a neoplasm containing at least one TMC zone will be analyzed to derive a present state neoplasmic profile of the neoplasmic mass comprising at least one of: a genomic profile, an epigenetic profile, a micro-RNA network profile, a proteomic profile. Techniques which may be used include, without limitation, single cell sequencing modalities. Ultimately, the goal is to make a determination of the relative master regulator complex network entropy of each of the one or more TMC zones within the neoplasm in order to determine the present-state driver zone of the neoplasmic mass, which is the region of the neoplasmic mass displaying the highest cellular master regulator complex network entropy.

Determination of the cellular master regulator complex network entropy of a given region of a neoplasmic mass may or may not necessarily be performed according to a deterministic fashion. Generally, it is to be understood that there are a number of ascertainable and/or potentially measurable factors which may contribute to the determination of the relative cellular master regulator complex network entropy of a given region of a neoplasmic mass. Such factors include, without limitation, an elevated cellular entropy, an elevated chromosomal instability, an elevated intratumor heterogeneity, a depressed transmembrane potential, an elevated resistance to microenvironmental noxions to growth, an elevating resistance to available pharmaceutical modalities, and an elevated resistance to survival inhibitory factors, among other potential factors. It is contemplated that some or all of these factors may be directly or indirectly ascertained as a consequence of identifying the present state neoplasmic profile, via known and future developed methods of analysis of biopsies of neoplasmic masses.

The determination of the relative cellular master regulator complex network entropy of a given region of a neoplasmic mass may be performed in a number of ways. It may be seen that one or more particular factors may be of more or less substantial importance in such a determination, or the confluence of a plurality of particular factors being present simultaneously. For example, it is contemplated that an algorithm may be developed according to the methods described herein where a measure of cellular master regulator complex network entropy may be derived as a particular value, considering as inputs the presence or absence of various of the above-mentioned factors, or particular values measured for the above-mentioned factors, or combinations thereof. In particular, it is contemplated that such an algorithm may include various weightings which may be accorded to certain factors or the combinatorial presence of certain factors. As such, it may be seen that this step of identifying the present-state driver zone may or may not necessarily be accomplished in a precise fashion (e.g. via a deterministic algorithm which procures an output figure) but alternatively may also be performed with less precision based upon the experience of one skilled in the fields of neoplastic genomics, polyomics, proteomics, etc., taking into consideration one or more of such factors.

Turning now to FIG. 1B, it is shown how therapies may operate to reduce the master regulator network entropy of the present-state driver zone, illustrated by the presence of the oval inclusions surrounding the master regulator and driver cluster of the TMC zone and inhibiting neoplastic growth. Such therapies may be any known or future developed cancer therapeutic, which may include, for example but without limitation, a programmable nano-machine, the delivery of a micro-RNA to the present-state driver zone via a non-pathogenic virus, modification of the transmembrane potential of the cells within the present-state driver zone, physical disruption of the present-state driver zone, a non-pathogenic virus containing crispr-CAS9 gene editing machinery, and the delivery of a gene of interest loaded into a liposome-like particle having an avidity for one or more surface receptors of one or more cells within the present-state driving zone. Generally speaking, the regulator of the present-state driver zone is to be understood as a good candidate for treatment via the herein described methods.

Turning now to FIG. 1C, various embodiments of programmable nano-machines are illustrated which may be utilized as therapeutics as described above. Such programmable nano-machines may be deployed into tumor mass to execute the task of appropriate reduction of master regulator network entropy of the present-state driver zone. There are a multiplicity of different ways to achieve this goal, and it may be seen that any such way in which the master regulator network entropy may be reduced may be appropriate. However, it may also be seen that it may be desirable for the programmable nano-machines to be customized for different malignancies, in different ways, and that a nearly infinite variety of different treatment modalities, delivery systems, or constructions or embodiments of the programmable nano-machines are possible.

For example, in case of glioblastoma multiforme, programmable nano-machines may get delivered intraoperatively or following surgical resection by painting the resection margins with a liquid containing the programmable nano machines. Likewise, programmable nano-machines, which are capable of modifying the master regulator complex network entropy and other distortions, may either get delivered intralesional or systemically, or in other ways. The reduction of the master regulator network entropy of the present-state driver zone may also be achieved via application of physical forces by the programmable nano-machine, such as via delivering a calculated level of electrostatic forces into the cells in the present-state driver zone. The reduction of the master regulator network entropy of the present-state driver zone may also be achieved via a delivery by the programmable nano-machine of an intracellular or extracellular component which may result in a modifications in genome, epigenome, protein, RNA, or micro-RNA compartments.

One more specific embodiment of a Programmable Nano-Machine, referred herein as Type A, may comprise nano-beads with precalculated electrostatic potential for delivery to the cell membranes of those cells within the present-state driver zone which function as regulators of that driver zone (RDZ), in order to increase their electrostatic force to the normal cell level. Another methodology could take advantage of pulsating waves directed to the RDZ cells. Following normalization of electrostatic force of RDZ cells, it is expected that genomic and epigenomic, as well as micro-RNA network of RDZ cells, would be modified accordingly. As such, the RDZ cells will go into freeze mode, and the forward evolutionary movement of the tumor mass may cease or slows down significantly. The time needed for the birth of another driver zone may then represent time where the patient's condition is not worsening, halting the progression of the disease and prolonging survival.

Another more specific embodiment of a Programmable Nano-Machine, referred herein as Type B, may be designed based on the information made available in developing the present state neoplasmic profile through polyomics and spatial genomics technology. Following identification of driver zone, Programmable Nano-Machine Type B may be delivered, with an affinity for surface receptors of identified present-state driver zone. Programmable Nano-Machine Type B, in this embodiment, is a non-pathogenic virus designed to introduce an intracellular component into the cells or the intracellular region around the identified present-state driver zone, such as a micro-RNA. If the micro-RNA is one which is normally missing, the absence of which constitutes a causative factor for malignancy of the present-state driver zone, then it may be expected that the present-state driver zone would lose its invasive features and the forward evolutionary movement, and would come to a standstill, thus arresting the progression of the disease and prolonging survival.

A further specific embodiment of a Programmable Nano-Machine, referred herein as Type C, may be operative to perform gene editing in the driver zone of interest. Such a Programmable Nano-Machine may utilize known or future developed mechanisms to be delivered to the site of the present-state driver zone, such as ligand-receptor interaction. The Type C programmable nano-machine may be, for example, a non-pathogenic virus loaded with crispr-CAS9 gene editing machinery. Alternatively, the Type C programmable nano-machine may be the gene of interest inserted into a liposome-like particle having an avidity for surface receptors on the cells in the zone of interest. It is, however, to be understood that all of the above described methods are to be understood as exemplary and not limiting on the scope or spirit of the present disclosure.

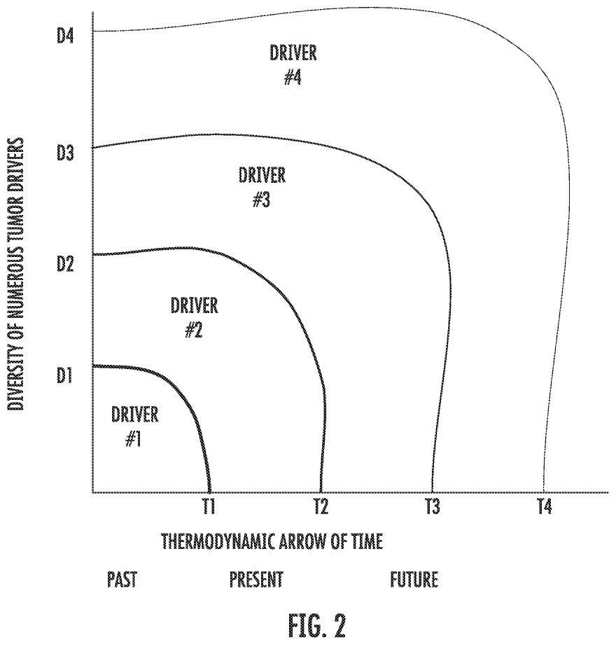

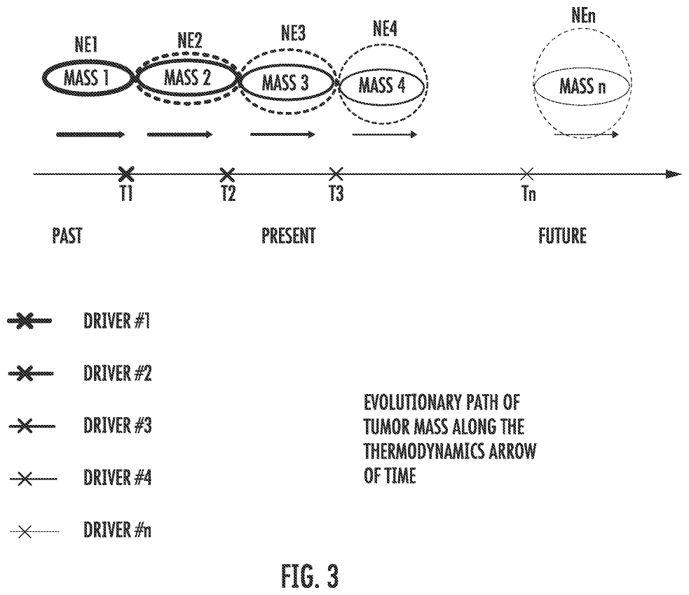

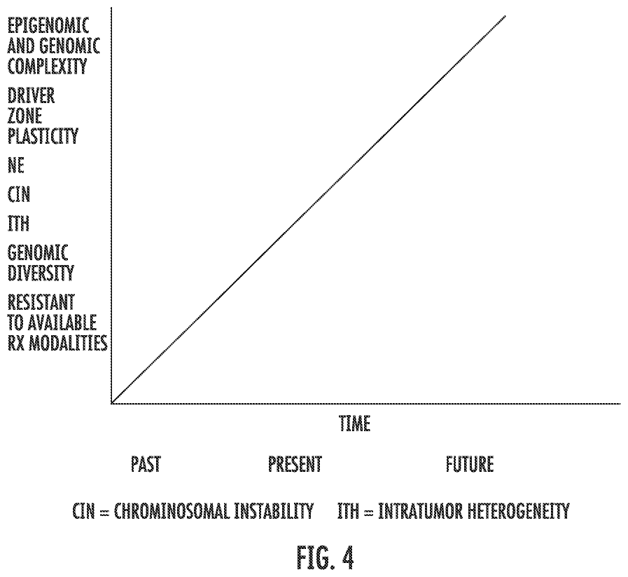

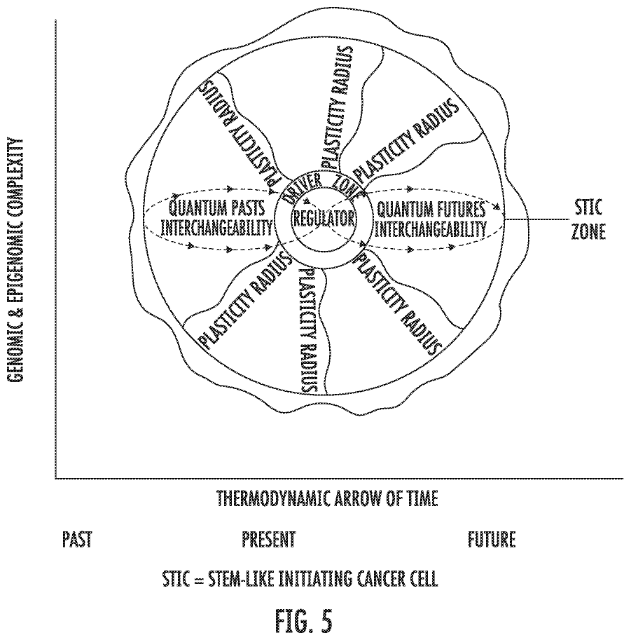

Turning now to FIGS. 2-5, it is important to understand the concept of the evolutionary procession within a tumor mass, and these figures each illustrate this procession in different fashions and according to various relevant factors which may contribute to the development of a new present-state driver zone having highest master regulator network entropy. In particular, it is to be understood that both in the presence and absence of treatment modalities, new drivers will tend to come into existence, where driver zones with higher master regulator network entropy will outcompete and supersede the existing present state driver zone to become the new present state driver zone, and thereafter become the dominant contributor to tumor growth. In this fashion, it may be seen that it will be important to be able to predict the future development of future-state driver zones.

In this fashion, it may be seen as desirable to utilize methods of evolutionary projection of the present-state neoplastic profile in order to project candidate future-state driver zones which may have an elevated propensity to develop into an active future-state driver zone, either spontaneously or in response to an administered therapy. Such candidate future-state driver zones may be within the master regulator and driver cluster of a TMC zone, or may be within a STIC regenerator zone. Techniques for evolutionary projection of the present-state neoplastic profile in order to project candidate future-state driver zones may, for example but without limitation, incorporate AI or machine learning features, and may be performed using any known or future developed methods of evolutionary projection in order to anticipate and identify one or more candidate future-state driver zones which may have an elevated propensity to develop into a projected active future-state driver zone comprising a region of the neoplasmic mass displaying the highest cellular master regulator complex network entropy.

Thereafter, once the projection of candidate future-state driver zones is performed, it may be seen that therapies may be administered to inhibit the potential of the candidate future-state driver zone to develop into an active future-state driver zone. Likewise, therapies may be administered to prophylactically reduce the master regulator network entropy of the projected active future-state driver zone, in the same fashion as the present-state driver zone was treated, as described above. Such therapies include all of those therapies as described above in relation to the treatment of the present-state driver zone, including, without limitation, therapies which comprise, or are administered by, a programmable nano-machine.

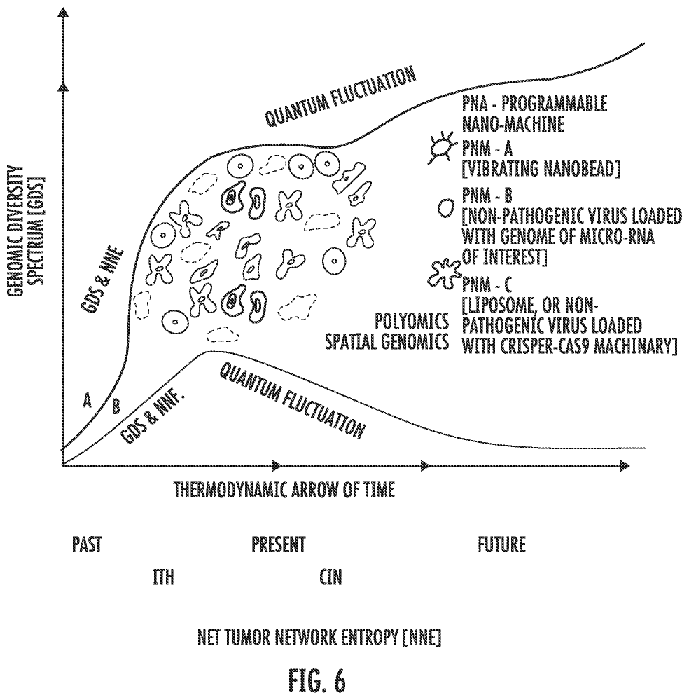

Turning now to FIG. 6, it may be seen how the processes of treatment described herein may treat the tumor not just by reducing the master regulator network entropy of the present-state driver zone, but also by inhibiting the potential of the candidate future-state driver zone to develop into an active future-state driver zone or by reducing the master regulator network entropy of the projected active future-state driver zone in a prophylactic fashion. In particular, it may be seen that Curve A may represent a tumor with high master regulator network entropy and a constantly increasing genetic diversity, and that intervention in the treatment fashions described herein may result in the conversion of Curve A to Curve B, which may be understood to represents a tumor with lower genomic diversity and lower master regulator network entropy. Due to the interference with the development of active future state driver-zones, either through inhibition of their formation or the prophylactic treatment thereof, it may be seen that not only may the increase of genetic diversity within the tumor mass be slowed, but the tendency of genetic diversity to increase may be reversed entirely.

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the exemplary embodiments.

What is claimed is:

1. A method for developing a spatial entropyomic profile of a neoplasm, the method comprising the steps of:
obtaining at least one biopsy from a neoplasmic mass;
analyzing the at least one biopsy to derive a present state neoplasmic profile of the neoplasmic mass comprising at least one of: a genomic profile, an epigenetic profile, a micro-RNA network profile, a proteomic profile;
identifying, from the present-state neoplasmic profile, a present-state driver zone of the neoplasmic mass, the present-state driver zone being the region of the neoplasmic mass displaying the highest cellular master regulator complex network entropy; and
identifying, from an evolutionary projection of the present-state neoplasmic profile, one or more candidate future-state driver zones of the neoplasmic mass, a candidate future-state driver zone comprising a region of the neoplasmic mass having an elevated propensity to develop into a projected active future-state driver zone comprising a region of the neoplasmic mass displaying the highest cellular master regulator complex network entropy;
wherein the height of the cellular master regulator complex network entropy of a given particular region of a neoplasmic mass is determined according to the magnitude of and confluence, relative to other regions of the neoplasmic mass, of a plurality of factors in that region including: elevated cellular entropy, elevated chromosomal instability, elevated intratumor heterogeneity, depressed transmembrane potential, elevated resistance to microenvironmental noxions to growth, elevating resistance to available pharmaceutical modalities, elevated resistance to survival inhibitory factors.

2. The method of claim 1, further comprising the step of administering a first therapy operative to reduce the master regulator network entropy of the present-state driver zone.

3. The method of claim 2, wherein the first therapy comprises, or is administered by, a programmable nano-machine.

4. The method of claim 2, wherein the first therapy comprises the delivery of a micro-RNA to the present-state driver zone via a non-pathogenic virus.

5. The method of claim 2, wherein the first therapy comprises modification of the transmembrane potential of the cells within the present-state driver zone.

6. The method of claim 2, wherein the first therapy comprises physical disruption of the present-state driver zone.

7. The method of claim 2, wherein the first therapy comprises a non-pathogenic virus containing crispr-CAS9 gene editing machinery.

8. The method of claim 2, wherein the first therapy comprises the delivery of a gene of interest loaded into a liposome-like particle having an avidity for one or more surface receptors of one or more cells within the present-state driving zone.

9. The method of claim 1, further comprising the step of administering a second therapy operative to inhibit the potential of the candidate future-state driver zone to develop into an active future-state driver zone.

10. The method of claim 9, wherein the second therapy comprises, or is administered by, a programmable nano-machine.

11. The method of claim 9, wherein the second therapy comprises the delivery of a micro-RNA to the candidate future-state driver zone via a non-pathogenic virus.

12. The method of claim 9, wherein the second therapy comprises modification of the transmembrane potential of the cells within the candidate future-state driver zone.

13. The method of claim 9, wherein the second therapy comprises physical disruption of the future-state driver zone.

14. The method of claim 9, wherein the second therapy comprises a non-pathogenic virus containing crispr-CAS9 gene editing machinery.

15. The method of claim 9, wherein the first therapy comprises the delivery of a gene of interest loaded into a liposome-like particle having an avidity for one or more surface receptors of one or more cells within the present-state driving zone.

16. The method of claim 1, further comprising the step of prophylactically administering a third therapy operative to reduce the master regulator network entropy of the projected active future-state driver zone.

17. The method of claim 16, wherein the third therapy comprises, or is administered by, a programmable nano-machine.

18. The method of claim 16, wherein the third therapy comprises the delivery of a micro-RNA to the projected active future-state driver zone via a non-pathogenic virus.

19. The method of claim 16, wherein the third therapy comprises modification of the transmembrane potential of the cells within the projected active future-state driver zone.

20. The method of claim 16, wherein the third therapy comprises physical disruption of the projected active future-state driver zone.

21. The method of claim 16, wherein the third therapy comprises a non-pathogenic virus containing crispr-CAS9 gene editing machinery.

22. The method of claim 16, wherein the third therapy comprises the delivery of a gene of interest loaded into a liposome-like particle having an avidity for one or more surface receptors of one or more cells within the projected active future-state driver zone.
